(19) United States
(12) Patent Application Publication
(10) Pub. No.: US 2008/0147078 A1
(43) Pub. Date: Jun. 19, 2008

(54) Method and Device for Determining Appropriate Spinal Rod Length

(76) Inventors: Thomas J. Francis, Cordova, TN (US); Kent M. Anderson, Memphis, TN (US)

Correspondence Address: MEDTRONIC;SPINAL AND BIOLOGICS, 2600 Sofamor Danek Drive, MEMPHIS, TN, 38132, US

(21) Appl. No.: 11/610,604

(22) Filed: Dec. 14, 2006

(51) Int. Cl. A61B 17/56

(52) U.S. Cl. 606/102

(57) ABSTRACT

A device and method for determining the appropriate length of rod to be placed between a first bone anchor assembly and a second bone anchor assembly in a spinal stabilization system is disclosed.

### BACKGROUND OF THE INVENTION

Spinal implant systems often provide a rod for supporting and positioning the spine in response to the specific problem being corrected by the implant and the patient's particular anatomy. Some systems include a support rod, bolts or screws secured to vertebrae, and a connector that securely engages the support rod to the connecting bolts/screws and allows the system to assume positions where the rod is maintained at various angles in response to the problem being corrected and the patient's particular anatomy.

Determination of the appropriate length of the rod is often accomplished by inserting trial instruments each having an end or a foot of different length to gauge the distance between the bolts or connectors. This trial and error process consumes operating time. Particularly if the rod will span more than two adjacent vertebra, the number of trial instruments required may be cumbersome.

### SUMMARY OF THE INVENTION

The present invention discloses a device for determining the appropriate length of a rod to be attached to a first bone anchor assembly and a second bone anchor assembly. The device includes a first member having a first end that is capable of being immobilized while the first end is in contact with the first bone anchor assembly. The device also includes a second member having a second end, the second member pivotally coupled to the first member and designed to pivot relative to the first member about a pivot point while the first member is immobilized such that the second end contacts the second bone anchor assembly. The device further includes an indicator capable of indicating the distance between the first end and the second end from which the appropriate length of a rod to be attached to the bone anchor assemblies is determined.

In another embodiment, a first sensor or marker is placed in contact with the first bone anchor assembly and a second sensor or marker is placed in contact with the second bone anchor assembly. The sensors or markers are in communication with an indicator capable of indicating the distance between the first sensor or marker and the second sensor or marker from which the appropriate length of a rod to be attached to the bone anchor assemblies is determined.

The present invention also discloses a method for determining the appropriate length of a rod to attach to a first bone anchor and a second bone anchor that includes temporarily immobilizing the first member having a first foot by engaging the first foot with the first bone anchor. Then, the second member having a second foot is moved relative to the first member, such that the second foot contacts the second bone anchor and obtaining an indication of the distance between the first foot and the second foot.

In one embodiment, the second member and the indicator are connected and are removably attachable to the first member. The first member may be a rod trial. The indicator is fixedly connectable to the first member and the second member is pivotally connectable to the indicator. The second member includes a needle, or scale arm, or the like that cooperates with the indicator to provide an indication of the distance between the end of the first member and the end of the second member. The distance between the end of the first member and the end of the second member when they are in contact with the first and second bone anchor assemblies, respectively, is indicative of the appropriate length of rod to be attached to the first and the second bone anchor assemblies.

Other embodiments are described herein and are apparent from the following discussion.

### DESCRIPTION OF THE DRAWINGS

In the accompanying drawings, which are incorporated in and constitute a part of this specification, embodiments of the invention are illustrated, which, together with a general description of the invention given above, and the detailed description given below, serve to exemplify the principles of this invention, wherein:

FIG. 1 illustrates a spinal rod connected between two bone anchor assemblies;

FIG. 2A illustrates front elevational view of an embodiment of the present invention;

FIG. 2B illustrates a side elevational view of an embodiment of the present invention;

FIG. 2C illustrates a rear elevational view of an embodiment of the present invention;

FIG. 3A illustrates front elevational view of an embodiment of the present invention;

FIG. 3B illustrates a side elevational view of an embodiment of the present invention;

FIG. 3C illustrates a rear elevational view of an embodiment of the present invention;

FIG. 4A illustrates front elevational view of an embodiment of the present invention;

FIG. 4B illustrates a side elevational view of an embodiment of the present invention;

FIG. 4C illustrates a rear elevational view of an embodiment of the present invention;

FIG. 5A illustrates a front left oblique view an embodiment of the present invention;

FIG. 5B illustrates a front right oblique view an embodiment of the present invention;

FIG. 5C illustrates a rear left oblique view an embodiment of the present invention;

FIG. 6 illustrates an embodiment of the present invention; and

FIG. 7 illustrates an embodiment of the present invention;

### DETAILED DESCRIPTION

The present invention discloses device, systems, and methods for determining the appropriate length of a rod to the attached to two bode anchor assemblies. The invention will be described in terms of the exemplary embodiments presented herein, but is limited only by the claims. For example, the following discussion the invention in terms of bone anchor assemblies attached to vertebra in a human spine as part of a spinal stabilization system; however, the invention may be used to determine the distance between two bone anchor assemblies in any part of a human or animal body.

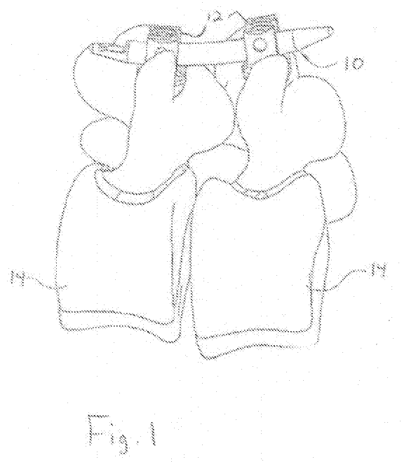

FIG. 1 illustrates a rod 10 placed between two bone anchor assemblies 12 affixed to two vertebrae 14. As illustrated, the vertebrae 14 are adjacent, but some applications require a rod 10 that extends between bone anchor assemblies 12 affixed to non-adjacent vertebrae 14. Conventionally, a trial and error process is used to determine the distance between two bone anchor assemblies 12. The present invention includes embodiments that minimize the trial and error necessary to determine the length of a rod 10 to be placed between two bone anchor assemblies 12 that are affixed to adjacent or non-adjacent vertebrae 14.

The bone anchor assemblies 12 are conventional and typically include bolts or screws secured to the vertebrae 14, and a connector that securely engages the rod 10 to the bolts or screws.

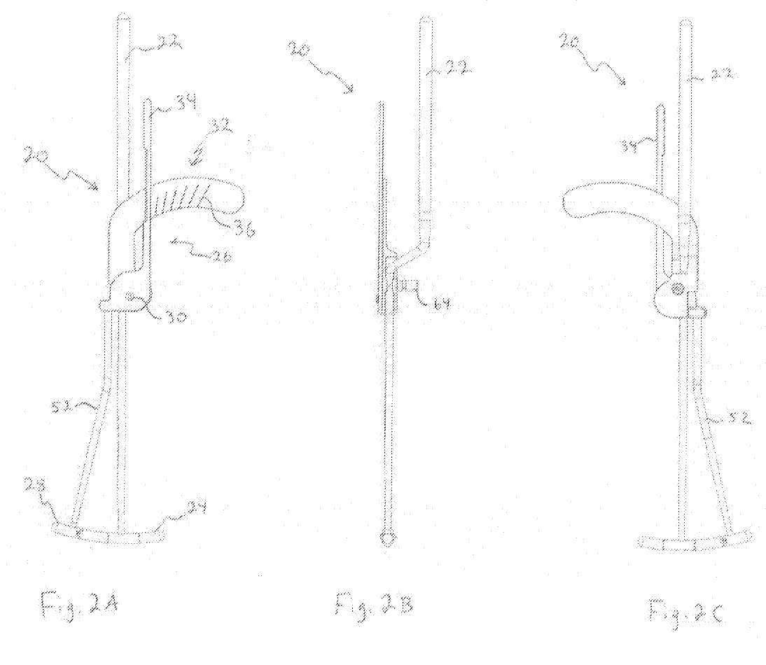

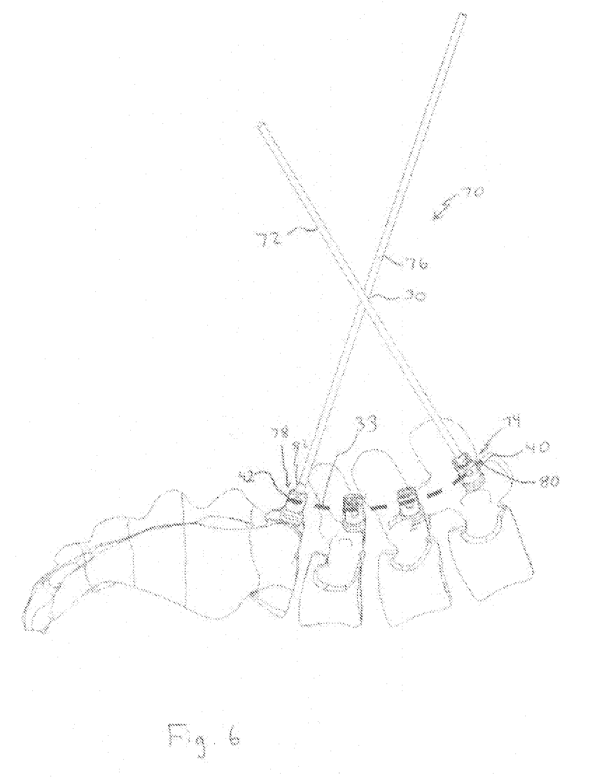

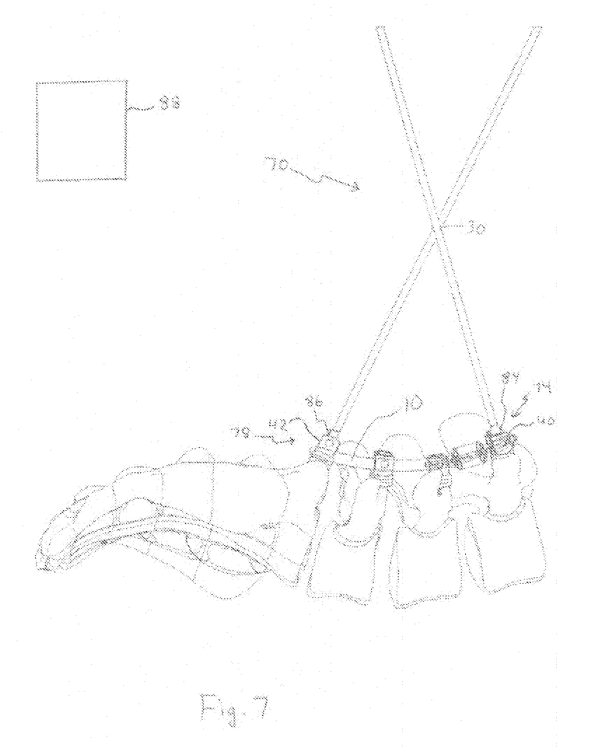

FIGS. 2A-C illustrate an embodiment of a device 20 for measuring the distance between two bone anchor assemblies 12 in which a first member 22 has a first end 24 and a second member 26 has a second end 28. The first member is pivotally coupled to the second member 26 at a pivot point 30. An indicator 32 is in communication with both the first member 22 and the second member 26 to provide an indication of the distance between the first end 24 and the second end 28. The distance between the first end 24 and the second end 28 is indicative of the length of a rod that would be placed between bone anchor assemblies 12 in contact with both first end 24 and second end 28. For example, the length of a rod could be at least about 5 mm longer than the distance between first end 24 and second end 28 to account for length necessary to engage attachment mechanisms to affix the rod to the bone anchor assemblies. Moreover, the direct distance between the bone anchor assemblies often requires adjustment because the rod may require an arcuate shape to accommodate the curvature of the spine between the vertebrae to which the rod will be attached. See FIG. 6 illustrating an exemplary are 33 and FIG. 7 illustrating an exemplary rod 10. Other adjustments are within the spirit and scope of the invention.

Typically, rods 10 are provided in fixed lengths. The indication of the distance between the bone anchor assemblies 12 will enable the user to select to appropriate rod length from the available fixed lengths rods based on criteria desired by the user. Alternatively, the indicator 32 provides an indication of the length of the rod to be placed between bone anchor assemblies 12 in contact with both first end 24 and second end 28 that would include adjustments, such as for extra length needed for affixation to the bone anchor assemblies 12.

In one embodiment, first end 24 is a first foot and second end 28 is a second foot, each foot having a shape substantially similar to the shape of a rod 10 that would be placed between the anchor assemblies 12. In this embodiment, the first foot 24 and the second foot 28 may be inserted into respective bone anchor assemblies 12 to fit as would the rod 10 to be placed into the bone anchor assemblies 12. The indication provided by the indicator 32 would be appropriately adjusted. Insertion of the first foot 24 and the second foot 28 into respective bone anchor assemblies 12 may also provide an indication of the alignment of the anchor assemblies 12. The first end, or foot, 24 and the second end, or foot 28 may have any shape.

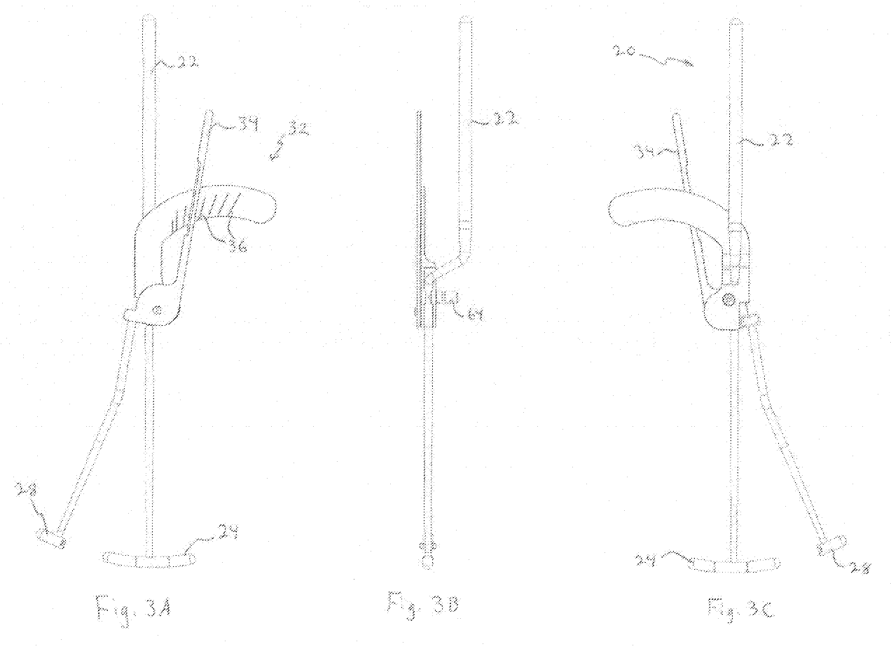

In one embodiment, the second member 26 includes a needle or scale arm 34 disposed on the opposite side of the pivot point 30 from the second end 28. The needle 34 communicates with the second end 28 such that movement of the second end 28 produces corresponding movement in the needle 34. For example, needle 34 and second end 28 are both integral with the second member 26 such that movement of the second end 28 may be produced by moving the needle 34. FIGS. 3A-C illustrate an embodiment of device 20 in which the second end 28 and the needle 34 have been moved relative to the first member 22 and indicator 32.

The needle 34 cooperates with the indicator 32 such that alignment of the needle 34 with reference to the indicator 32 will provide indication of the distance between the first end 24 and the second end 28. In this embodiment, indicator 32 is fixed with respect to the first member 22 so that movement of the second end 28 and the corresponding movement of the needle 34 provides differing alignment of the needle 34 with the indicator 32 depending on the distance between the first end 24 and the second end 28.

In one embodiment, the indicator 32 includes gradation marks 36 indicative of the distances that could be present between bone anchor assemblies 12. Thus, the alignment of the needle 34 with the gradation marks 36 would correspond to the distance between the two particular bone assemblies 12 for each use of the device 20.

In operation, the device 20 is inserted into an opening (not shown) in the body of a patient. The first member 22 is temporarily immobilized by engagement or contact of the first end 24 with a first bone anchor assembly 40. The second member 26 is moved relative to the immobilized first member 22 such as by pivotal movement with respect thereto about the pivot point 30 such that the second end 28 contacts a second bone anchor assembly 42. The device 20 may be designed such that the path of second end 28 approximates the arcuate shape of the rod 10 to be inserted between the bone anchor assemblies 12.

As illustrated in FIG. 3A, movement of the second end 28 corresponds to movement of the needle 34 with respect to the indicator 32. The indicator 32 is fixed relative to the first member 22. The position of the needle 34 relative to the gradation marks 36 on the indicator 32 provides an indication of the distance between the first end 24 and the second end 28, and, by correlation, between the first bone anchor assembly 40 and the second bone anchor assembly 42. From this indication, the appropriate length of rod 10 to be placed between the first bone anchor assembly 40 and the second bone anchor assembly 42 can be determined.

Alternatively, the second member 26 is temporarily immobilized by engagement or contact of the second end 28 with the second bone anchor 42. The first member 22 is then moved relative to the immobilized second member 26.

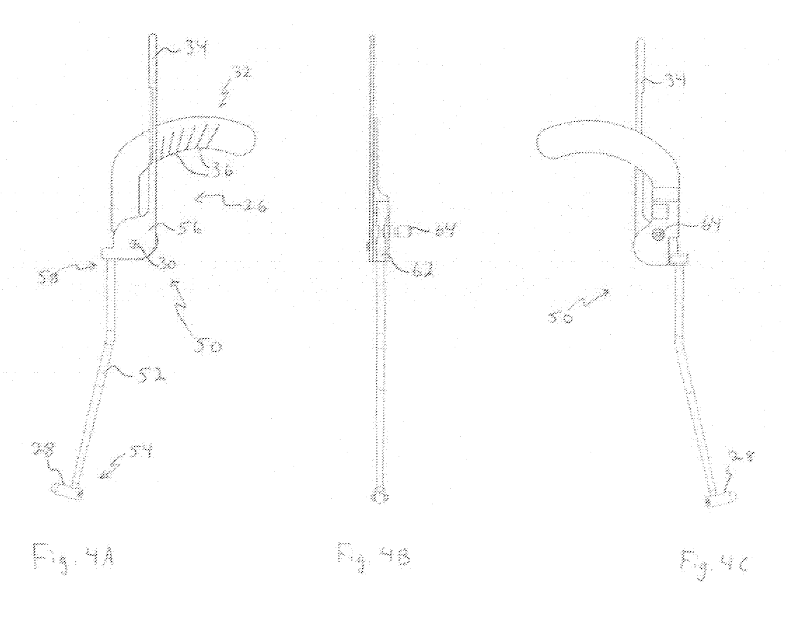

In one embodiment of the present invention, as illustrated in FIGS. 4A-C, rod gauge 50 is separate from first member 22. In this embodiment, the rod gauge 50 is removably attachable to first member 22. Rod gauge 50 includes second member 26 and indicator 32. The second member 26 and the indicator 32 are attached or connected at pivot point 30. This attachment may be a permanent attachment, such as by a rivet, bolt, or other permanent mechanical fastener, or a removable attachment, such as with a screw, cotter pin, or other removable fastener without departing from the spirit or scope of the invention. The attachment provides for pivotal movement of second member 26 with respect to indicator 32.

Second member 26 includes needle or scale arm 34, elongated member 52 having second end, or foot, 28 disposed at the distal end 54, and medial portion 56. In this embodiment, elongated member 52 is attached or connected to medial portion 56 at proximal end 58. Needle 34 also is attached or connected to the medial portion 58, with pivot point 30 disposed within the medial portion 56 such that needle 34 and elongated member 52 are disposed on opposite sides thereof. In this arrangement, movement of needle 34 in one direction results in corresponding movement of second end 28 in the opposite direction, as can be seen from FIG. 4A. For example, if needle 34 is moved to the right, as illustrated in FIG. 4A, then second end 28 will correspondingly move to the left.

Indicator 32 includes gradation marks 36, a slot 62, and a fastener 64, which can be seen in FIGS. 4A-C, and 5A-C. Gradation marks 36 indicate a scale, such as a distance scale to indicate distance between the first end 24 and second end 28, or a rod length scale, which is based on the distance between first end 24 and second end 28, as discussed above. In one embodiment, the gradation marks correspond to rod length and are provided in 10 mm increments, such as between 20 mm and 120 mm. Other increments may be selected without departing from the spirit and scope of the invention.

Slot 62 is designed to accommodate first member 22 such that fastener 64 will fasten indicator 32 to first member 22. Thus, when second member 26 pivots about pivot point 30, second member 26 pivots with respect to first member 22 as well as indicator 32, because indicator 32 and first member 22 are fixed with respect to each other.

In one embodiment, fastener 64 is a locking screw operable by finger pressure alone. Preferably, fastener 64 provides for removal of first member 22 from fixed engagement with indicator 32. Other fasteners, such as clamps, etc., may be used without departing from the spirit and scope of the invention.

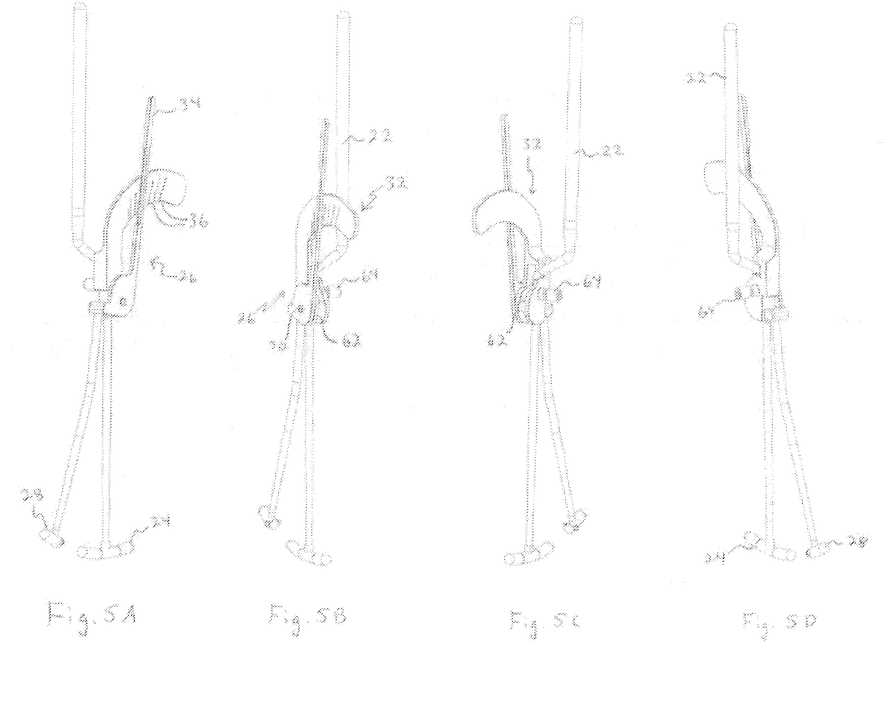

In operation, the rod gauge 50 is attached to first member 22 by placing first member 22 into slot 62 and tightening or fastening fastener 64 to engage and secure first member 22 to rod gauge 50. See FIGS. 4 and 5. First member 22 may be a rod trial included in the instrument set.

First end 24 and second end 28 are placed into an opening in the body of a patient such that first end 24 engages or contacts first bone anchor assembly 40 and first end 24 is temporarily immobilized. The user then moves needle 34 sufficiently to pivot second member 26 relative to first member 22 and to bring second end 28 into contact with the second bone anchor assembly 42. The position of needle 34 relative to gradation marks 36 on indicator 32 when the second end 28 contacts the second bone anchor assembly 42 provides an indication of the distance between first bone anchor assembly 40 and second bone anchor assembly 42. The appropriate length of rod 10 to be placed between first bone anchor assembly 40 and second bone anchor assembly 42 may then be determined based on the indicated distance. Alternatively, as discussed above, the gradations may indicate an appropriate length of rod 10 without providing an explicit indication of the distance between first bone anchor assembly 40 and second bone anchor assembly 42.

If desired, the rod gauge 50 may be removed from engagement/attachment with the first member 22, or rod trial, after the appropriate length of rod is determined. Removal of the rod gauge 50 from the first member 22, or rod trial, may facilitate subsequent sterilization of the first member 22 and the rod gauge 50 for reuse in another procedure.

In another embodiment, a device 70 for measuring the distance between two bone anchor assemblies 40, 42 includes first member 72 having first end 74 and second member 76 having second end 78. See FIG. 6. First end 74 includes first marker 80 and second end 78 includes second marker 82. First marker 80 and second marker 82 are markers/devices designed to be identified, to communicate with, and/or to be used in computer- and/or image-guided surgical navigation systems and techniques.

A number of different types of surgical navigation systems have been described that include indications of the positions of medical instruments and patient anatomy used in medical or surgical procedures. For example, U.S. Pat. No. 5,383,454 to Bucholz; PCT Application No. PCT/US94/04530 (Publication NO. WO 94/24933) to Bucholz; and PCT Application No. PCT/US95/12894 (Publication No. WO 96/11624) to Bucholz et al., the entire disclosures of which are incorporated herein by reference, disclose systems for use during a medical or surgical procedure using scans generated by a scanner prior to the procedure. Surgical navigation systems typically include tracking means such as, for example, an LED array on the body part, LED emitters on the medical instruments, a digitizer to track the positions of the body part and the instruments, and a display for the position of an instrument used in a medical procedure relative to an image of a body part. U.S. Pat. No. 6,021,343, to Foley et al., which is incorporated herein by reference in its entirety, discloses a trackable medical instrument for use in computer assisted image guided medical and surgical navigation systems that generate images during medical and surgical procedures. U.S. Pat. No. 6,226,548 to Foley et al., which is incorporated herein by reference in its entirety, discloses a percataneous registration apparatus and computer assisted surgical navigation system for use during spinal surgery, U.S. Pat. No. 7,063,705, to Young, et. al., which is incorporated herein by reference in its entirety, discloses methods and devices for fluoroscopic registration of the spinal column that also allow registration of more than one vertebral level for use during spinal surgery.

First marker 80 and second marker 82 are, for example, opaque with respect to one or more of these computer- or image based navigation systems, or otherwise in communication with an output device in the navigation systems that is remote from the first marker 80 and the second marker 82. As a result, the appropriate length of rod to be placed between first bone anchor assembly 40 and second bone anchor assembly 42 can be identified and determined via the selected navigation system based on the distance between the first marker 80 and second marker 82 indicated by detection by and/or communication with the selected navigation system. The navigation system may indicate on an output device (not shown) the distance between the first marker 80 and the second marker 82 or the output device may indicate the appropriate length of rod to be placed between first bone anchor assembly 40 and second bone anchor assembly 42.

Specific modifications to the selected navigation system and output device will be apparent to one of ordinary skill in the art without undue experimentation, depending on the specific configuration, software, etc., of the navigation system selected. Determination of the appropriate rod length via the navigation system may include accommodation for the desired curvature of the rod to be inserted, depending on the geometry of the rod to be used and the geometry of the anatomy of the particular patient.

In one embodiment of the device 70 of the present invention illustrated in FIG. 7, first end 74 is provided with a first sensor 84 and second end 78 is provided with a second sensor 86. First sensor 84 and second sensor 86 are devices in circuit communication with a sensor output device 88. Sensor output device 88 is an indicator and includes a processing unit to process the signals received from the first sensor 84 and the second sensor 86 to determine the distance between the first sensor 84 and the second sensor 86. Output device 88 provides an output indication to the user of the distance between first sensor 84 and second sensor 86 or an output indication indicating the appropriate length of rod to be placed between first bone anchor assembly 40 and second bone anchor assembly 42 when first end 74 is placed in contact with first bone anchor assembly 40 and second end 78 is placed in contact with second bone anchor assembly 42.

First sensor 84 and second sensor 86 may be in circuit communication with each other to provide an indication of the distance between the two sensors to the output device 88 for determination of appropriate rod length. In one embodiment, first sensor 84 and second sensor 86 are transmitters, transmitting their positions to the output device 88 with or without regard to the position of the other sensor. In one embodiment, output device 88 is a computer, such as a personal computer, or other microprocessor-based device having a screen capable of providing an output to a user.

“Circuit communication” as used herein indicates a communicative relationship between devices. Direct electrical, electromagnetic, and optical connections and indirect electrical, electromagnetic, and optical connections are examples of circuit communication. Two devices are in circuit communication if a signal from one is received by the other, regardless of whether the signal is modified by some other device. For example, two devices separated by one or more of the following—amplifiers, filters, transformers, optoisolators, digital or analog buffers, analog integrators, other electronic circuitry, fiber optic transceivers, or oven satellites—are in circuit communication if a signal from one is communicated to the other, even though the signal is modified by the intermediate device(s). As another example, an electromagnetic sensor is in circuit communication with a signal if it receives electromagnetic radiation from the signal. As a final example, two devices not directly connected to each other, but both capable of interfacing with a third device, e.g., a CPU, are in circuit communication.

In one embodiment, first sensor 84 and second sensor 86 are removably applied to first end 74 and second end 78, respectively, such that first sensor 84 and second sensor 86 may be removed from first end 74 and second end 78. This removable application may be by adhesive, magnetic attraction, or any other method of removable attachment without departing from the spirit and scope of the invention. This will facilitate sterilization of the device 70 without the need to attempt to sterilize the sensors. The sensors are then disposed of to eliminate the need for sterilization for reuse. In one embodiment, first sensor 84 and second sensor 86 are disposable.

First member 72 and second member 76 may be, but need not be, pivotally connected at pivot point 30. Pivot point 30 may be provided at any point along the length of first member 72 and second member 76, such as near the respective midpoints or and end of first member 72 and second member 76. The location of the pivot point 30 may be selected without departing from the spirit or scope of the invention.

Alternatively, first member 72 and second member 76 are unconnected and the first end 74 and second end 78 are placed into contact with bone anchor assemblies 40, 42 such that the associated markers 80, 82 or sensors 84, 86 are in circuit communication, and/or in circuit communication with an output device, to indicate the appropriate length of rod to be placed between the bone anchor assemblies 40, 42.

In another embodiment, markers 80, 82 or sensors 84, 86 are placed in contact with the bone anchor assemblies 40, 42 without an associated first member 72 pivotally connected to a second member 76. For example, the sensors or markers may be temporarily screwed directly into the bone anchor assemblies. The sensors or markers may be temporarily, or permanently, affixed to the bone anchor assemblies in any manner, such a mechanical affixation, adhesive fixation, or the like, without departing from the spirit and scope of the invention.

There are multiple combinations possible of the various features of the illustrated embodiments and such features may be combined in any way to achieve the desired device and method for determining appropriate spinal rod length.

While the present invention has been illustrated by the above description of embodiments, and while the embodiments have been described in some detail, it is not the intention of the applicants to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicants' general or inventive concept. Additionally, although some features, concepts, or aspects of the invention may be described herein as being a preferred arrangement or method, such description is not intended to suggest that such feature is required or necessary unless expressly so stated.

We claim:

1. A device for determining the appropriate length of a rod to be attached to a first bone anchor assembly and a second bone anchor assembly, the device comprising:
a. a first member comprising a first end, the first member capable of being immobilized while the first end is in contact with the first bone anchor assembly;
b. a second member comprising a second end, the second member pivotally coupled to the first member and designed to pivot relative to the first member about a pivot point while the first member is immobilized such that the second end contacts the second bone anchor assembly; and
c. an indicator capable of indicating at least one of the distance between the first end and the second end and the appropriate length of a rod to be attached to the bone anchor assemblies.

2. The device of claim 1, wherein the first member comprises a rod trial.

3. The device of claim 1, wherein the first end comprises a first foot and the second end comprises a second foot, wherein the first foot and the second foot comprise a shape substantially similar to the rod to be attached to the bone anchors.

4. The device of claim 1, wherein the second member comprises a needle disposed opposite the pivot point from the second end, and further wherein the needle is capable of movement corresponding to the movement of the second end.

5. The device of claim 4, wherein the needle cooperates with indicator to indicate the distance between the first end and the second end.

6. The device of claim 5, wherein the indicator is fixed relative to the first member.

7. The device of claim 6, wherein the indicator comprises gradations and the position of the needle relative to the gradations indicates the distance between the first end and the second end.

8. The device of claim 1 wherein the second member is removable coupled to the first member.

9. The device of claim 1, wherein the indicator is connected to the first member.

10. The device of claim 1, wherein the indicator is removable connected to the first member.

11. The device of claim 1, wherein the indicator is connected to the second member.

12. The device of claim 1, further comprising a first sensor associated with the first end and a second sensor associated with the second end.

13. The device of claim 12, wherein the indicator is in circuit communication with the first sensor and the second sensor.

14. The device of claim 13, wherein the indicator is remote from the first sensor and second sensor.

15. The device of claim 1, wherein the indicator comprises gradations.

16. A device for determining the distance between two bone anchor assemblies, the device comprising:
a. a first elongated member comprising a first foot at an end thereof;
b. a gauge coupled to the first elongated member, the gauge comprising
i. a second elongated member comprising a second foot at an end thereof;
ii. a scale arm connected to the second elongated member;
iii. a scale in mechanical communication with the scale arm, the scale comprising indication marks;
wherein the second member and the scale arm are disposed for pivotal movement relative to the first elongated member such that movement of the scale arm causes corresponding movement of the second elongated member and wherein the location of the second foot determines the position of the scale arm relative to the scale.

17. The device of claim 16, wherein the position of the scale arm relative to the scale provides indication of the distance between the two bone anchor assemblies.

18. The device of claim 17, wherein the distance between two bone anchor assemblies corresponds to an appropriate length of a rod to be secured to the two bone anchor assemblies.

19. The device of claim 18, wherein at least one of the first foot and the second foot is substantially cylindrical and has substantially the same diameter as the rod to be secured to the two bone anchor assemblies.

20. The device of claim 16, wherein the first elongated member is a rod trial device.

21. The device of claim 16, wherein the gauge is removably coupled with the first elongated member.

22. The device of claim 21, wherein the gauge further comprises a slot designed to engage a portion of the first elongated member and a locking screw capable of locking the gauge to the first elongated member after engagement of the slot with the first elongated member.

23. The device of claim 16, wherein the gauge is permanently attached to the first elongated member.

24. The device of claim 16, wherein the scale arm is integral with the second elongated member.

25. The device of claim 16, wherein the indication marks provide indication of the distance between the two bone anchor assemblies.

26. The device of claim 25, wherein the alignment of the scale arm relative to the indication marks on the scale when the first foot and the second foot are engaged with respective bone anchor assemblies provides an indication of the distance between the two bone anchor assemblies and provides an indication of an appropriate length of a rod to be affixed to the two bone anchor assemblies.

27. The device of claim 16, wherein the second foot comprises at least one flange.

28. The device of claim 27, wherein the flange is designed to contact a bone anchor assembly to limit the movement of the second elongated member.

29. The device of claim 16, wherein the bone anchor assemblies comprise pedicle screws.

30. The device of claim 16, wherein the scale is designed for coupling with the first elongated member, and the second elongated member and the scale arm are pivotally attached to the scale.

31. A rod gauge designed for attachment to a first member, comprising:
a. a second member comprising a first end;
b. a scale arm mechanically affixed to the second member;
c. a scale in mechanical communication with the scale arm, the scale comprising indication marks;
wherein the second member and the scale arm are designed for pivotal coupling to the first member such that movement of the scale arm causes corresponding movement of the second member and wherein the position of the first end determines the position of the scale arm relative to the scale.

32. The rod gauge of claim 31, wherein the scale is designed for affixation to the first member, and the second member and the scale arm are pivotally attached to the scale.

33. The rod gauge of claim 31, wherein the scale further comprises a slot designed to engage a portion of the first member and a locking screw capable of locking the scale to the first member after engagement of the slot with the first member.

34. The rod gauge of claim 31, wherein the first member is a rod trial.

35. A device for determining the length of a rod to be attached to a first bone anchor assembly and a second bone anchor assembly, the device comprising:
a. a first member for contacting the first bone anchor assembly;
b. a second member pivotally coupled with the first member and designed to contact the second bone anchor assembly;
c. means to actuate pivotal movement of the second member relative to the first member until the second member contacts the second bone anchor assembly; and
d. means for indicating the length of the rod to be attached to the first and second bone anchors based on the distance between corresponding portions of the first member and the second member that contact the first bone anchor and the second bone anchor.

36. The device of claim 35, wherein the second member comprises a proximal end and a distal end with a pivot point therebetween.

37. The device of claim 36, wherein the distal end is designed to contact the second bone anchor assembly.

38. The device of claim 37, wherein the proximal end comprises means for providing indication of the distance between the distal end and an end of the first member that contacts the first bone anchor assembly.

39. The device of claim 38, wherein the distal end cooperates with a graduated scale to provide indication of the distance between the distal end and the end of the first member.

40. The device of claim 36, wherein the distal end comprises a sensor that cooperates with a corresponding sensor on the first member to provide indication of the distance between the distal end and the end of the first member.

41. The device of claim 40, wherein the means for indicating the length of the rod comprises an indicator in circuit communication with the sensors.

42. The device of claim 35, wherein the second member is removably coupled to the first member.

43. A rod gauge designed for attachment to a first member, comprising:
a. a second member comprising a foot;
b. means for attaching the second member to the first member to enable pivotal movement of the second member relative to the first member; and
c. means for determining the distance between the foot and a predetermined portion of the first member.

44. The rod gauge of claim 43, wherein the means for attaching the second member to the first member comprises means for removable attaching the second member to the first member.

45. The rod gauge of claim 44, wherein the means for attaching the second member to the first member comprises a slot in the second member to engage the first member and a locking screw to attach the second member to the first member.

46. The rod gauge of claim 43, wherein the second member comprises a proximal end and a distal end with a pivot point therebetween.

47. The device of claim 46, wherein the proximal end comprises means for providing indication of the distance between the distal end and an end of the first member.

48. The device of claim 47, wherein the distal end cooperates with a graduated scale to provide indication of the distance between the distal end and the end of the first member.

49. The device of claim 47, wherein the distal end comprises a sensor that cooperates with a corresponding sensor on the first member to provide indication of the distance between the distal end and the end of the first member.

50. A device for determining the appropriate length of rod to be placed between a first bone anchor assembly and a second bone anchor assembly, the device comprising:
a. a first member having a first end capable of being temporarily immobilized while in contact with the first bone anchor assembly;
b. a first communication device in association with the first end;
c. a second member having a second end capable of contacting the second bone anchor assembly, the second member pivotally attached to the first member; and
d. a second communication device in association with the second end;
wherein the first communication device and the second communication device are capable of communicating with a remote output device.

51. The device of claim 50, further comprising a remote output device capable of communication with the first communication device and the second communication device to determine and display at least one of the distance between the first communication device and the second communication device and the appropriate length of rod to be placed between the first bone anchor assembly and the second bone anchor assembly based on the distance therebetween.

52. The device of claim 51, wherein the remote output device is a component of a selected image-guided surgical navigation system.

53. The device of claim 52, wherein the first and second communication devices are opaque relative to the navigation system such that the position of the first and second communication devices can be detected and determined by the navigation system.

54. The device of claim 50, wherein the first and second communication devices comprise markers.

55. The device of claim 51, wherein the remote output device comprises a processor and is in circuit communication with the first communication device and the second communication device.

56. The device of claim 50, wherein the first and second communication devices are sensors.

57. The device of claim 50, wherein the first and second communication devices are transmitters.

58. The device of claim 50, wherein the second member is removable attachable to the first member.

59. A method for determining the appropriate length of a rod to attach to a first bone anchor and a second bone anchor, the method comprising:
temporarily immobilizing a first member having a first end by engaging the first end with the first bone anchor;
moving a second member having a second end relative to the first member, such that the second end contacts the second bone anchor;
obtaining an indication of the appropriate length of the rod to attach to the first bone anchor and the second bone anchor based on the distance between the first end and the second end.

60. The method of claim 59, wherein moving the second member comprises pivotal movement relative to the first member.

61. The method of claim 59, wherein the first member and the second member are elongated.

62. The method of claim 59, wherein moving the second member comprises moving a scale arm attached to the second member such that the scale arm moves relative to a scale that is fixed to the first member.

63. The method of claim 62, wherein the position of the scale arm relative to gradations on the scale provides indication of the distance between the first end and the second end.

64. The method of claim 59, wherein the first bone anchor and the second bone anchor are used in a spinal stabilization system.

65. A method for determining the appropriate length of a rod for attachment to a first bone anchor and a second bone anchor, the method comprising:
attaching a rod gauge to a rod trial for pivotal movement therebetween about a pivot point, the rod gage comprising a foot and a scale arm disposed on opposite sides of the pivot point and a scale fixed relative to the rod trial;
temporarily immobilizing the rod trial by engaging the rod trial with the first bone anchor;
pivotally moving the foot and scale arm such that the foot contacts the second bone anchor and the scale arm moves relative to the scale;
obtaining an indication of the appropriate rod length from the position of the scale arm relative to the scale.

66. The method of claim 65 further comprising removing the rod gauge from the rod trial.

67. The method of claim 65, wherein the first bone anchor and the second bone anchor are used in a spinal stabilization system.

68. The method of claim 65, wherein the scale comprises gradations and the alignment of the scale arm relative to the gradations provides an indication of the appropriate rod length.
